(19) United States
(12) Patent Application Publication
(10) Pub. No.: US 2007/0250099 A1
(43) Pub. Date: Oct. 25, 2007

(54) Multiple Tip Lancet

(76) Inventors: Bruce A. Flora, Elkhart, IN (US); Joseph E. Ruggiero, Goshen, IN (US)

Correspondence Address: NIXON PEABODY LLP, 161 N. CLARK STREET, 48TH FLOOR, CHICAGO, IL, 60601, US

(21) Appl. No.: 11/632,800

(22) PCT Filed: Jul. 20, 2005
(86) PCT No.: PCT/US05/25664

§ 371 Date: Jan. 18, 2007

Related U.S. Application Data
(60) Provisional application No. 60/589,357, filed on Jul. 20, 2004.

(51) Int. Cl. A61B 17/32

(52) U.S. Cl. 606/181

(57) ABSTRACT

A lancet comprises a body and a shaft. The body has an end face and a bore. The shaft is secured to the body and extends through the bore such that a distal section of the shaft extends outwardly from the end face of the body. The distal section has an axial slot that extends through the distal end of the shaft to form a first and a second protrusion. The first and second protrusions each have a spike on the end thereof.

### TECHNICAL FIELD

The present invention relates to a medical device for extracting bodily fluids. More particularly, the present invention relates to a lancet having multiple penetrating tips.

### BACKGROUND

Those who have irregular blood glucose concentration levels are medically required to regularly self-monitor their blood glucose concentration level. An irregular blood glucose level can be brought on by a variety of reasons including an illness such as diabetes. An estimated 18 million people are afflicted with diabetes in the United States alone. A diabetic patient typically monitors their blood glucose concentration level to determine whether the level is too high or too low and whether any corrective action, such as administering insulin or other medication, is necessary to bring the level back within a normal range. The failure to take corrective action can have serious implications. When blood glucose levels drop too low—a condition known as hypoglycemia—a person can become nervous, shaky, and confused. That person's judgment may become impaired and that person may eventually pass out. A person can also become very ill if their blood glucose level becomes too high—a condition known as hyperglycemia. Both conditions, hypoglycemia and hyperglycemia, are both potentially life-threatening emergencies. As a result, a diabetic may require frequent sampling of his or her blood glucose—typically several times per day.

Lancets are used to puncture the skin and draw capillary blood for these diagnostic tests. Lancets typically consist of a shank portion with a blade or spike at the distal end. The blade or spike is sharp and adapted to pierce a patient's skin so as to sever capillaries and provide blood for testing. Due to the sharpness of the spike, lancets are typically provided with a removable shield to cover the blade or spike when not in use to protect the patient and other users from inadvertent skin punctures. Lancets are also typically loaded into spring-loaded injectors that propel the lancet forward with enough force to puncture the skin.

Due to the frequent testing required, a diabetic may develop calluses at the puncture sites. These calluses may prevent sufficient blood flow to obtain a sample. In addition, due to sensitive nerve endings in the fingertip area, lancet punctures sometimes induce a significant amount of pain. Attempts have been made to provide a lancet that reduces the amount of pain a patient experiences. Most developments in lancet design have concentrated upon different needle grind angles or on smaller needle gages (i.e., diameters). However, the use of smaller needle gages may not generate a puncture that is large enough to provide a sufficient sample. What is needed is a lancet design that produces enough blood to be tested, while reducing the associated pain.

One way of obtaining a sufficient sample while reducing the associated pain is by producing multiple, closely spaced, shallow punctures. It has been found experimentally that a person may perceive a puncture by two closely spaced needles as a single puncture. Multiple needles, however, have been overlooked due to the economics of manufacturing large quantities cost effectively or a belief that multiple needles would increase pain. For example, aligning the tips of multiple needles so that each tip is at the proper angle and the same height as the other tips requires additional manufacturing steps and cost. Further, the smaller needle gages that are used in multiple needle designs may cause some tips to bend as the needles pierce a calloused area on a diabetic patient's skin. What is needed is an improved lancet having multiple tips for extracting blood.

### BRIEF SUMMARY

Accordingly, an advantage of the present invention is to provide a new and improved multiple tip lancet. In particular, advantages of the present invention are to provide a lancet that provides for a unitary shaft with multiple tips that reduce the associated pain a patient experiences by allowing for a shallower puncture depth with adequate sample volume, that is cost-efficient to manufacture so that it may be used as a disposable, one-time-use device, and which overcomes the problems or limitations discussed above.

In accordance with these and many other advantages of the present invention, the present invention is embodied in a lancet comprising a body and a shaft. The body has an end face and a bore. The shaft is secured to the body and extends through the bore such that a distal section of the shaft extends outwardly from the end face of the body. The distal section has an axial slot that extends through the distal end of the shaft to form a first and a second protrusion. The first and second protrusions each have a spike on the end thereof.

According to a second aspect of the present invention, a lancet comprises a body and a U-shaped shaft. The body has an end face and a bore. The shaft is secured the body and extends through the bore. The U-shaped shaft has a first end substantially aligned with a second end in a spaced apart configuration. The first and second end are each beveled such that a spike is formed on each of the first and said second ends.

A third aspect of the present invention is embodied in a method of making a lancet comprising the acts of: providing a shaft; forming an axial slot in a distal end of said shaft so that a first protrusion and a second protrusion are formed; beveling the first and second protrusions so that the first and second protrusions each have a spike; providing a body with a bore; and securing the shaft to the body so that the distal end of the shaft extends from the bore.

A fourth aspect of the present invention is embodied in another method of making a lancet comprising the steps of: providing a shaft with a first end and a second end; bending the shaft into a “U”-shape so as to align the first end of the shaft with the second end of the shaft in a spaced apart configuration; beveling the first end to form a first spike; beveling the second end to form a second spike; providing a body with an end face; and securing the shaft to the body so that the first and second ends extend outwardly from the end face.

### BRIEF DESCRIPTION OF THE DRAWINGS

FIG. 1 is a cross section view of one embodiment of the lancet assembly of the present invention;

FIG. 2 is a detail perspective view of the lancet tip of FIG. 1;

FIG. 3 is a top perspective view of the lancet tip of FIG. 1;

FIG. 4 is side perspective view of the lancet tip of FIG. 1;

FIG. 5 is a cross section view of another embodiment of the lancet assembly of the present invention;

FIG. 6 is an end view of the lancet tip of FIG. 5;

FIG. 7 is a cross section view of another embodiment of the lancet assembly of FIG. 5;

FIG. 8 is a detail perspective view of the lancet tip of FIG. 5;

FIG. 9 is a graphical depiction of the sample volumes obtained using different numbers of needles; and

FIG. 10 is a graphical depiction of the pain a patient experiences using different numbers of needles.

### DETAILED DESCRIPTION OF THE DRAWINGS AND THE PRESENTLY PREFERRED EMBODIMENTS

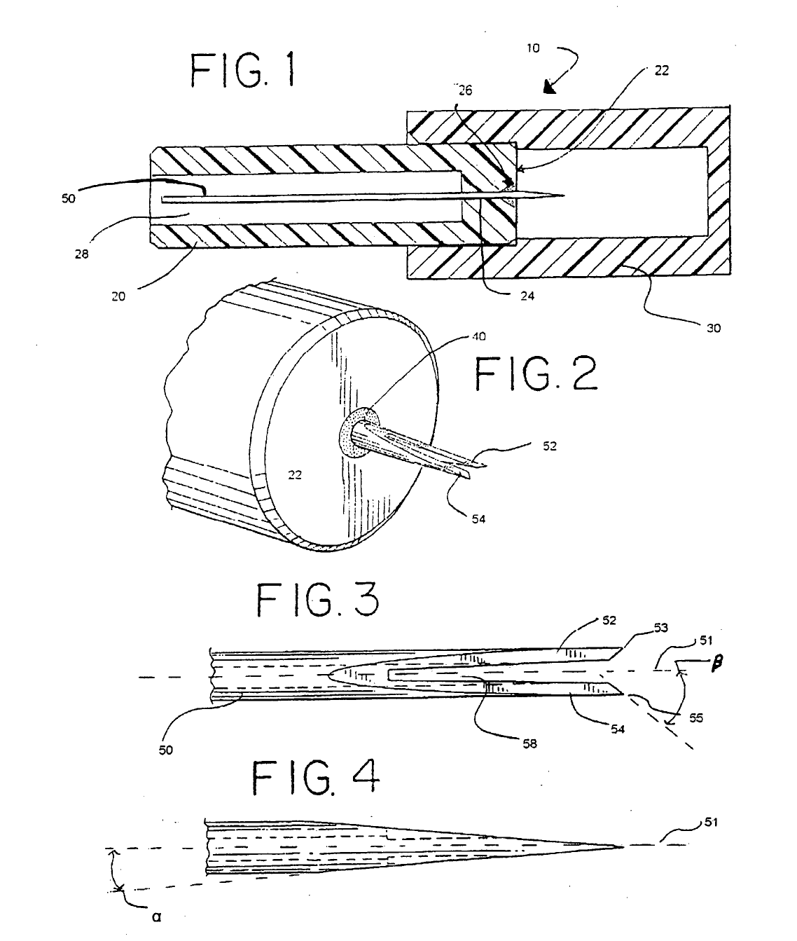

Referring now more specifically to the drawings, therein is disclosed a multiple tip lancet assembly generally designated by the reference numeral 10 and embodying the present invention. As seen in FIG. 1, one embodiment of a multiple tip lancet assembly 10 comprises a plastic body 20, a protective cover 30, and a shaft 50. FIGS. 2-3 illustrate the multiple protrusions of shaft 50 that form the multiple tip lancet, with a first protrusion 52 and a second protrusion 54 formed on the distal section of the shaft 50.

Plastic body 20 may be formed as a molded plastic cylinder, with an end face 22 and a bore 24 located along its axis. Shaft 50 is secured to the body 20 such that a distal section of the shaft extends through the end face 22. A countersink 26 may also be formed along bore 24 on the end face 22 on plastic body 20. An ultraviolet adhesive 40 may be applied to countersink 26 to secure shaft 50 to plastic body 20 and prevent or inhibit any relative rotation or axial movement of the shaft 50 relative to the body 20.

Alternately, and as seen in FIG. 7, shaft 70 may be molded into plastic body 20. A concave or bent portion 76, or some other type of projection on shaft 70, may provide a mechanical interference such that shaft 70 is secured to body 20. Other methods of attaching shaft 70 to body 20 may also be used, such as welding.

Referring to FIG. 1, the assembly of shaft 50 into plastic body 20 may be facilitated by forming a counterbore 28 at the end opposite end face 22 on plastic body 20. To make lancet assembly 10 inexpensive and disposable, plastic body 20 and cover 30 may be molded from any suitable plastic such as polyethylene, polycarbonate, acetal, or ABS (acrylonitrile-butadiene-styrene). Body 20 is sized to allow for use in existing lancing devices, as described below. For example, body 20 may be formed as a cylinder with a diameter of 0.250 inches and an axial length of 0.900 inches.

As best seen in FIGS. 2-4, first protrusion 52 and second protrusion 54 may be formed on shaft 50 in several ways. In the embodiment shown in these figures, shaft 50 may be formed from a length of 27 gage (or some other gage) stainless steel hypodermic needle tubing. For example, shaft 50 may have an outer diameter of 0.016 inches and an inner diameter of 0.008 inches, although tubing with other sizes may be used as well. The first and second protrusions 52, 54 on shaft 50 may be made through the following series of steps: First, as seen in FIG. 3, a chamfer at an angle β relative to a central axis 51 is applied to the distal end of shaft 50. Preferably, a chamfer of approximately forty five degrees by 0.004 inches may be applied to either the inside diameter or outside diameter of shaft 50 such that a hollow beveled tip is formed on shaft 50. However, chamfers of other angles and depths may also be used.

Second, first protrusion 52 and second protrusion 54 are formed by making a slot 58 that is cut into a distal end of shaft 50 along the central axis 51, preferably 0.004 inches wide by 0.075 inches long. Slot 58 controls the flash produced by the grinding operations in the third step described below. Slot 58 may be formed through a grinding operation. Alternately, slot 58 may be made by a laser or other suitable metal cutting means capable of forming a finely machined channel in a tube. If desired, additional slots may be formed along the distal end of shaft 50 along central axis 51 to form additional protrusions. For example, a slot may be made perpendicular to slot 58 along a distal end of central axis 51 of shaft 50, thus forming four separate protrusions.

Third, and as seen in FIG. 4, a distal end of shaft 50 has opposing bevel grinds on either side of slot 58 to form a first spike 53 and a second spike 55. In forming the bevel grinds, shaft 50 is retained at an angle a of approximately 5 degrees relative to the central axis of shaft 50 while a grinding wheel is translated from left to right in FIG. 4, thereby forming the sharp pointed end on first spike 53 and second spike 55 that slope relative to the central axis 51. Any suitable clamping means may be used to firmly hold shaft 50 while the distal end is ground therein. Alternatively, the grinding wheel may be non-translatable and shaft 50 is supported in translatable gripping means whereby shaft 50 can be translated in an opposite direction into the grinding surface of the wheel so as to form a pointed end. The grinding wheel (or the shaft 50) is rotated one hundred eighty degrees to repeat the grinding operation on the opposite side of the distal end of shaft 50. Alternately, the bevels on either side of slot 58 may be made by laser or other suitable metal cutting means capable of forming a sharp bevel on a tube. By applying the chamfer in the first step, described above, to either the inside diameter or outside diameter, the orientation of first spike 53 and second spike 55 may be controlled such that the cutting surfaces are facing either towards each other or away from each other. Additional surfaces of the shaft 50 may also be ground to alter the exterior shape of the shaft 50.

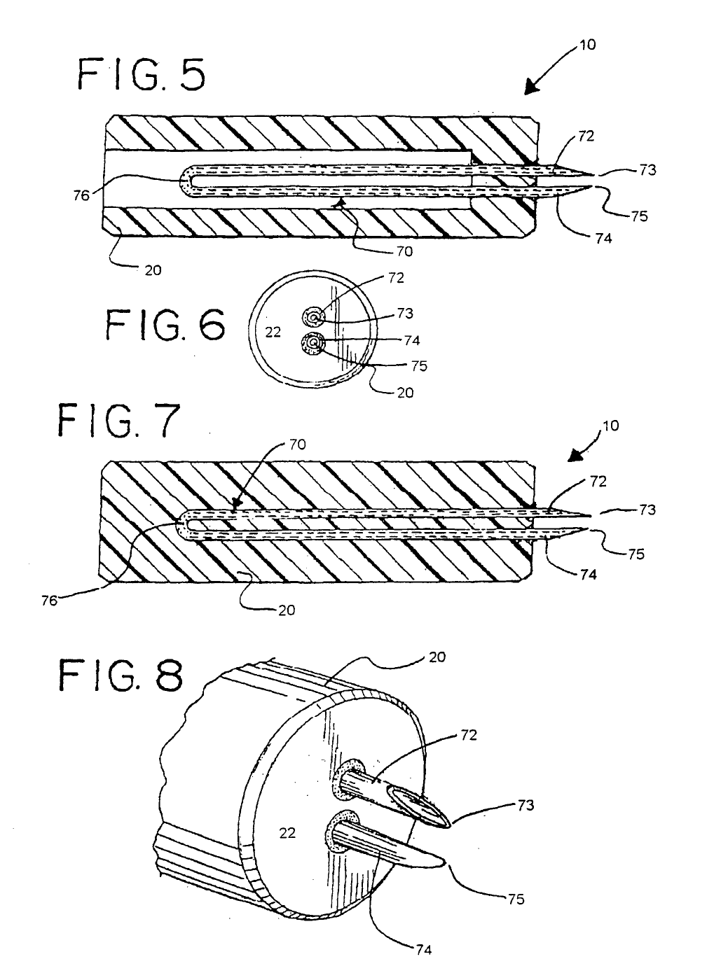

Alternately, in embodiments shown in FIGS. 5-8, shaft 70 is shaped such that both ends of shaft 70 are substantially aligned with each other in a spaced apart configuration and form a first protrusion 72 and a second protrusion 74 with concave or bent portion 76 in between. In the embodiments shown in FIGS. 5-8, shaft 70 is preferably made from a length of 30 gage (or some other gage) stainless steel hypodermic needle tubing. Shaft 70 may be shaped by first bending shaft 70 until both ends are approximately parallel and substantially aligned with each other in a spaced apart configuration, preferably forming a general “U”-shape or “U”-shape. For example, first protrusion 72 and second protrusion 74 may be aligned with a 0.030 inch gap between their axes, although they may be aligned with larger or smaller gaps as well.

Second, the ends of shaft 70 may be trimmed such that first protrusion 72 and second protrusion 74 are approximately the same length. This trimming operation may be performed by a grinding operation, a laser, a saw, or any other suitable metal cutting means. For example, after the trimming operation, both first protrusion 72 and second protrusion 74 may be approximately 1.000 inches long, although they may be trimmed to other lengths as well. Third, the ends on first protrusion 72 and second protrusion 74 are bevel ground such that a first spike 73 and a second spike 75 are formed with substantially sharp points, as described above. As described above in connection with the embodiment shown in FIGS. 1-4, shaft 70 may then be attached to body 20. For example, after the shaft 70 is attached to body 20, first protrusion 72 and second protrusion 74 may extend from body 20 a length of 0.060 inches.

In either of the embodiments shown in FIGS. 1-4 or FIGS. 5-8, first spike 53, 73 and second spike 55, 75 on shaft 50, 70 may then be coated with a silicone or other biocompatible lubricant to ease insertion into a patient's skin. First spike 53, 73 and second spike 55, 75 are shaped such that they are capable of puncturing skin to obtain a drop of blood for a sample.

Lancet assembly 10 is adapted for a sterile, single-use application. Protective cover 30 serves as a removable shield to cover the first spike 53, 73 and second spike 55, 75 on shaft 50, 70 when the lancet assembly 10 is not in use, protecting the patient and others from inadvertent skin punctures.

In operation, lancet assembly 10 may be used to obtain a sample by first removing protective cover 30 and then driving first spike 53, 73 and second spike 55, 75 on shaft 50, 70 into a patient's skin until the skin in punctured. Alternately, lancet assembly 10 may used with a lancing device that has a spring-loaded mechanism that drives the spikes into a patient's skin to a pre-selected depth. Examples of such devices are the Ascensia® MICROLET® Adjustable Lancing Device or the MICROLET® VACULANCE® Lancing Device available from Bayer Corporation of Elkhart, Ind., United States of America. It should be noted that a lancet assembly 10 of the present invention may be used with a lancing device that incorporates components that are similar in design and/or function as described in U.S. Pat. No. 5,954,738, issued Sep. 21, 1999, and entitled Blood Sampling Device with Lancet Damping System. The contents of this patent are hereby incorporated by reference to avoid unnecessary duplication of the description of similar components.

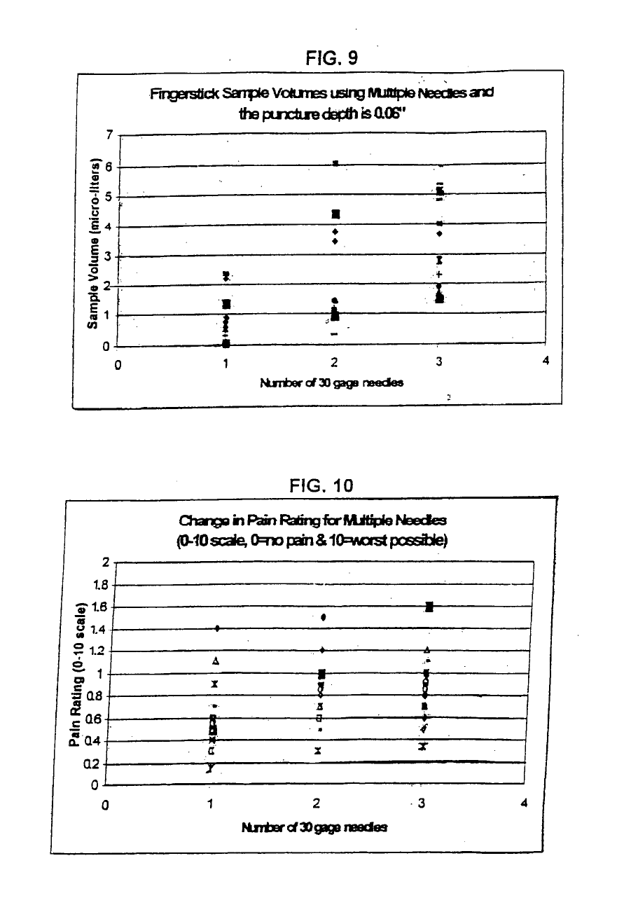

When using lancet assembly 10 with such lancing devices described above, a patient may use a shallower depth setting than what would be used for a single spike lancet and still draw a sufficient sample volume. As seen in FIG. 9, while holding the puncture depth and needle gage constant, larger sample volumes were obtained using multiple needles as opposed to using single needles. This is understood because the multiple needle punctures increase both the probability that a single capillary will be cut and the number of capillaries that are cut. In addition, the larger sample volumes obtained in FIG. 9 were obtained without a significant increase in perceptible pain, as seen in the results in FIG. 10. In FIG. 10, pain ratings were measured using a slide algometer, which is a slide rule-type device for communicating pain sensations. After a patient feels the lancet puncture, the patient pulls a slide exposing an underlying red surface. The length of the exposed surface is proportional to the pain experienced. It is reported on a scale of 0-10, with 0.1 increments for a total of 100 divisions. In the present invention, the patient perceives the multiple punctures as a single puncture, and by reducing the depth setting, the patient would have a decreased perception of pain.

The sample may then be applied to a glucose test strip or test sensor used with a blood glucose monitor to obtain blood glucose levels. One example of such a test strip is the Ascensia® AUTODISC® with ten test strips available from Bayer Corporation of Elkhart, Ind., United States of America. Examples of blood glucose monitors are the Ascensia® BREEZE® Blood Glucose Monitoring System or the Ascensia® DEX® 2 Blood Glucose Monitoring System, also available from Bayer Corporation of Elkhart, Ind., United States of America.

Thus, there has been disclosed in accordance with the present invention, a multiple tip lancet that that provides a reduction in the pain inflicted on a patient when piercing the patient's skin in accordance with the present invention. With a unitary shaft with multiple protrusions and spikes, the present invention allows for small, rigid, closely-spaced multiple tips formed to close tolerances. It also allows for reduced costs—both in raw materials and assembly. Further, the multiple tips of the present invention are easily aligned with each other and allow all of the tips to be securely attached to the body.

Although the invention has been described and illustrated with reference to specific illustrative embodiments thereof, it is not intended that the invention be specific limited to those illustrative embodiments. Those skilled in the art will recognize that variations and modifications can be made without departing from the spirit of the invention. For example, whereas two sharp spikes are shown on the distal end of the shaft, the shaft may be machined such that more than two spikes are formed. Alternately, the shape of the sharp spikes may be modified such that different cutting angles are formed by different grinds or bevels. Moreover, the shaft or the plastic body may be arranged to have a different configuration in cross section, such as a square or oval, solid or hollow. In addition, the sequence of steps used to make the lancets may be performed in any order. It is therefore intended to include within the invention all such variations and modifications that fall within the scope of the appended claims and equivalents thereof.

1. A lancet comprising:
a body having an end face and a bore; and
a shaft secured to said body and extending through said bore such that a distal section of said shaft extends outwardly from said end face, said distal section having an axial slot extending through a distal end of said shaft that forms a first and a second protrusion, said first and said second protrusions each having a spike.

2. The lancet of claim 1 further comprising a protective cap removably covering said first and said second protrusions.

3. The lancet of claim 2 wherein said first and said second protrusions are beveled so that a sharp point is formed on each of said spikes of said first and said second protrusions.

4. The lancet of claim 1 wherein said shaft is a stainless steel tube.

5. The lancet of claim 4 wherein said shaft is a hypodermic needle tube.

6. The lancet of claim 5 wherein said shaft is secured to said bore by an ultraviolet adhesive.

7. The lancet of claim 5 wherein said first and second protrusions are coated with a lubricant.

8. The lancet of claim 5 wherein said shaft is constructed from tubing sized from 25 gage to 29 gage.

9. A method of making a lancet comprising the acts of:
providing a shaft;
forming an axial slot in a distal end of said shaft so that a first protrusion and a second protrusion are formed;
beveling said first and said second protrusions so that said first and said second protrusions each have a spike;
providing a body with a bore; and
securing said shaft to said body so that said distal end of said shaft extends from said bore.

10. The method of claim 9 further comprising covering said first and said second protrusions with a removable protective cap.

11. The method of claim 9 further comprising coating said first and said second protrusions with a lubricant.

12. A lancet for use in a blood testing device comprising:
a body having an end face and a bore;
a shaft secured to said body and extending through said bore such that a distal section of said shaft extends outwardly from said end face, said distal section having an axial slot extending through a distal end of said shaft that forms a first and a second protrusion, said first and said second protrusions each having a spike capable of puncturing skin to obtain a drop of blood; and
a protective cap removably covering said first and said second protrusions.

13. A lancet comprising:
a body having an end face and a bore; and
a shaft secured to said body and extending through said bore, said shaft having a first end substantially aligned with a second end in a spaced apart configuration, said first and second ends being beveled such that a spike is formed on each said first and said second ends.

14. The lancet of claim 13 further comprising a protective cap removably covering said first and said second protrusions.

15. The lancet of claim 13 wherein said shaft is generally “U”-shaped.

16. A method of making a lancet comprising the acts of:
providing a shaft with a first end and a second end;
bending said shaft so as to align said first end of said shaft with said second end of said shaft in a spaced apart configuration;
beveling said first end to form a first spike on said first end;
beveling said second end to form a second spike on said second end;
providing a body having an end face; and
securing said shaft to said body so that said first end and said second end extend outwardly from said end face.

17. The method of claim 16 further comprising trimming said first end such that it is substantially the same length as said second end.

18. The method of claim 16 further comprising covering said first and said second spikes with a removable protective cap.

19. The method of claim 16 further comprising coating said first and said second spikes with a lubricant.

20. The method of claim 16 wherein during the act of bending said shaft, said shaft is bent to form a general “U”-shape.

21. The method of claim 16 wherein during the act of securing said shaft to said body, said body being molded around said shaft.

22. A method of using a lancet with a lancing device to sample blood glucose, the lancing device having an adjustable depth mechanism to control the depth of a puncture, the method comprising the acts of:
providing a lancet assembly, wherein the lancet assembly has a body including an end face and a bore, wherein the lancet assembly has a shaft, said shaft secured to said body and extending through said bore such that a distal section of said shaft extends outwardly from said end face, said distal section having an axial slot extending through a distal end of said shaft that forms a first and a second protrusion, said first and said second protrusions each having a spike capable of puncturing skin to obtain a drop of blood;
loading said lancet assembly into said lancing device;
adjusting said adjustable depth mechanism on said lancing device to a preselected value; and
activating said lancing device to drive said spikes into a patient's skin to a depth proportional to said preselected value.
